(19) United States
(12) Patent Application Publication
(10) Pub. No.: US 2025/0222213 A1
(43) Pub. Date: Jul. 10, 2025

(54) SYSTEMS AND METHODS FOR PREDICTING AND QUANTIFYING DELIVERED AMPLITUDE, FREQUENCY, AND MEAN AIRWAY PRESSURE TO PATIENTS RECEIVING HIGH FREQUENCY VENTILATION AND BUBBLE CPAP

(71) Applicant: Cortex Manufacturing Inc., Lake Stevens, WA (US)

(72) Inventors: Sunil SALUJA, Seattle, WA (US); Bharath MODAYUR, Seattle, WA (US); Andrew Douglas JONES, Lake Stevens, WA (US)

(21) Appl. No.: 19/008,998

(22) Filed: Jan. 3, 2025

Related U.S. Application Data
(60) Provisional application No. 63/618,106, filed on Jan. 5, 2024.

(51) Int. Cl. A61M 16/00

(52) U.S. Cl. CPC A61M 16/0051; A61M 16/0003; A61M 16/026; A61M 2205/18; A61M 2209/02; A61M 2230/63; A61M 2240/00

(57) ABSTRACT

A ventilation quality sensor system can include a ventilation quality sensor comprising one or more accelerometers; one or more processors; and one or more computer-readable recording media that store instructions that are executable by the one or more processors to configure the ventilation quality sensor system to: (i) access acceleration data obtained via the one or more accelerometers of the ventilation quality sensor; and (ii) process the acceleration data using one or more artificial intelligence modules to generate ventilation quality output or predicted respiratory support device setting output.

### CROSS-REFERENCE TO RELATED APPLICATIONS

This application claims priority to U.S. Provisional Patent Application No. 63/618,106, filed on Jan. 5, 2024, and entitled “SYSTEMS AND METHODS FOR PREDICTING AND QUANTIFYING DELIVERED AMPLITUDE, FREQUENCY, AND MEAN AIRWAY PRESSURE TO PATIENTS RECEIVING HIGH FREQUENCY VENTILATION AND BUBBLE CPAP”, the entirety of which is incorporated herein by reference for all purposes.

### STATEMENT REGARDING GOVERNMENT-SPONSORED RESEARCH

N/A

### BACKGROUND

Neonatal intensive care units (NICUs) in the United States provide crucial, often lifesaving care to over 300,000 infants annually, out of nearly 4 million births. Approximately 9-18% of newborns require NICU care, and 10-20% of these NICU admissions experience respiratory distress syndrome (RDS), affecting around 40,000 infants annually in the US alone. These numbers are amplified worldwide. After the discovery and development of therapeutic surfactants, mortality due to this syndrome has been significantly reduced in the US. Before the field of neonatology existed (the pre-surfactant era), the neonatal mortality rate (NMR) was 35 per 1000 live births. Since then, medical advances have decreased NMR in a linear fashion to its current rate of ˜4 per 1000 live births. Despite these striking improvements, pre-term birth remains the world's number one cause of newborn deaths (almost 30%) and RDS continues to be the leading cause of death in premature infants globally.

Bubble continuous positive airway pressure (bCPAP) and High Frequency Ventilation (HFV) are common methods for respiratory management of neonates with RDS. bCPAP is an affordable, low-tech, non-invasive ventilation solution. Use of bCPAP is associated with decreased incidence of chronic lung disease and reduced overall mortality. As a low-tech, high impact solution, however, bCPAP is susceptible to significant practice variation. Suboptimal bCPAP administration can contribute to silent respiratory failure, bronchopulmonary dysplasia, and even mortality. bCPAP failures often occur silently since bubbling loss or infant mask dislodgement does not trigger alarms, and infants can quietly develop worsening hypercapnia and hypoxemia. Under current methods, assessment of optimal bubbling quality relies on subjective human judgement, making it prone to errors and interobserver variability.

For extremely premature infants and those with RDS who do not respond to bCPAP or conventional ventilation, high-frequency ventilation (HFV) is a critical treatment option. HFV's effectiveness, however, depends on the subjective assessment of the ‘chest wiggle factor,’ making it equally susceptible to inter-observer variability. Failure to promptly recognize changes in chest wiggle factor can lead to immediate and life-threatening complications.

Additionally, operational settings for HFV devices are not based on a uniform set of parameters. Different brands of HFV devices often use different sets of parameters (though the basic operating principle of the ventilation technique remains the same). For instance, for one brand of HFV devices, ventilation support delivery may be determined using respiratory rate (RR), peak inspiratory pressure (PIP), and positive end-expiratory pressure (PEEP) parameters, whereas another brand of HFV devices may use mean airway pressure (MAP), amplitude, frequency, and/or inspiratory time parameters. Physicians often encounter difficulty when transitioning to different healthcare establishments that use different HFV devices with different operational setting/parameter frameworks. Such variability in operational setting frameworks further contributes to variability in the assessment of ventilation support delivery and/or the effectiveness of HFV interventions.

The subject matter described herein is not limited to embodiments that operate only in environments or contexts such as those described above. Rather, this background is only provided to illustrate one exemplary technology area where some embodiments described herein may be practiced.

### BRIEF DESCRIPTION OF THE DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope. The disclosure will be described and explained with additional specificity and detail through the use of the accompanying drawings in which:

FIG. 1 illustrates example components of example systems that may comprise or implement the disclosed embodiments;

FIG. 2 illustrates a ventilation quality sensor connected to a life-like model of a premature infant that is connected to a high frequency oscillatory ventilator;

FIG. 3 illustrates a ventilation quality sensor connected to a water/bubble chamber of a bCPAP device.

FIG. 4 illustrates a schematic of an artificial intelligence (AI) module that is configured to receive 3-channel acceleration data and output a classification label.

FIG. 5 illustrates a table depicting support vector regression predictions of ventilator variables based on raw acceleration data and features extracted from raw acceleration data.

FIG. 6 illustrates a conceptual representation of an example architecture for a ventilation quality sensor system.

FIGS. 7 through 9 illustrate example flow diagrams depicting acts associated with the disclosed subject matter.

### DETAILED DESCRIPTION

Before describing various embodiments of the present disclosure in detail, it is to be understood that this disclosure is not limited to the parameters of the particularly exemplified systems, methods, apparatus, products, processes, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the particular example terminology used herein is for the purpose of describing the embodiments and is not necessarily intended to limit the scope of the claimed invention.

As noted above, bCPAP and HFV are common methods for respiratory management of neonates with RDS. However, suboptimal administration of bCPAP poses significant challenges. bCPAP failures are not infrequent, and can result in respiratory failure, the need for re-intubation, bronchopulmonary dysplasia, and even mortality. bCPAP failures occur due to the silent loss of CPAP function either through the loss of bubbling or infant mask dislodgement, leading to worsening hypercapnia and hypoxemia. The maintenance of adequate bubbling can help alleviate these failures, but the assessment of optimal bubbling remains a subjective measurement that is highly susceptible to human error and interobserver variability. Bedside caregivers must continually assess the degree of bubbling. Little or no bubbling suggests a loss of respiratory support. Too much bubbling can suggest condensation in the tubing or a dramatic increase in lung compliance, which, if not identified early, can pose risks to the patient. If bCPAP is dislodged, there is often no alarm since bCPAP systems are mostly low-tech, closed circuit, non-digital solutions. Some bCPAP units have begun to use pressure sensors, which may provide some benefit, but they do not measure bubbling, which is an important determinant of bCPAP effectiveness. As strain on nurse staffing increases, and as migration to private room care in NICUs continues, the introduction of human error will only increase.

For extremely premature infants and those with RDS who do not respond to bCPAP or conventional ventilation, high frequency ventilation (HFV) is a critical treatment option. HFV is available in most Level III or higher NICUs. HFV operation is also vulnerable to human error due to inter-observer variability of a critically important, yet highly subjective parameter known as the “chest wiggle factor” or, simply, “chest wiggle.” Since HFV is used in infants who are critically ill, caring for these infants is exceptionally challenging, with high risks of mortality from underlying illnesses like sepsis and pneumonia. Respiratory care of an infant on HFV is highly dependent on the subjective, observational assessment of chest wiggle by the bedside caregiver. Each change of shift brings a new observer with a new set of eyes and a potentially varied level of training. The failure to recognize any significant change in chest wiggle over the course of a hospital shift can result in immediate and life-threatening complications.

At least some disclosed embodiments are directed to utilizing accelerometer technology and artificial intelligence (AI) to quantify ventilation being delivered to a subject (e.g., an infant) via bCPAP or HFV. Such embodiments can effectively replace subjectivity in assessing optimal ventilation delivery with a quantitative measurement using real-time sensor data.

At least some embodiments provide a ventilation quality sensor that can be used to (i) objectively measure the quality of ventilation being delivered to a subject (e.g., an infant) via bCPAP and/or HFV devices (e.g., HFOV devices), (ii) objectively measure chest wiggle in HFV settings, and/or (iii) estimate patient oxygenation or CO2 levels to enable the prediction of respiratory decompensation. A ventilation quality sensor, as disclosed herein, may comprise an AI-driven sensor platform that can leverage off-the-shelf, inexpensive sensor hardware (e.g., accelerometers) to provide continual, objective assessment of ventilation quality. A ventilation quality sensor, according to the disclosed subject matter, can be similar in size and form to existing sensor devices such as transcutaneous CO2 or ECG sensors. In use, a ventilation quality sensor can be affixed directly to the subject's chest (e.g., an infant's chest) or, alternatively, a bCPAP bubble chamber. Sensor data acquired via the ventilation quality sensor may be processed by one or more AI modules/models, which can enable prediction of bCPAP and HFV failures before they occur. Such predictive functionality can be a valuable tool for the bedside nurse or other practitioner in the management of respiratory care of the infant/subject.

The ventilation quality sensors and/or associated systems/techniques described herein can advance respiratory care by quantifying the effectiveness of ventilation mechanisms in delivering optimal respiratory support to a significant subgroup of neonates requiring critical care. For instance, a ventilation quality sensor, as described herein, can be used to predict silent respiratory decompensation through non-invasive assessment of vibrations of the chest wall (in the case of HFV). Providing an objective basis for determining ventilation quality delivered via HFV devices (e.g., objectively quantifying or classifying chest wiggle) can address difficulties associated with the use of different operational settings/parameters by different HFV devices (e.g., the ventilation quality output can be agnostic toward the setting/parameter framework). As another example, a ventilation quality sensor, as described herein, can be used to reduce the incidence of critical respiratory emergencies by monitoring the bubble chamber for infants on bCPAP. Such functionality can lead to reduced long-term complications such as the incidence of bronchopulmonary dysplasia. Furthermore, by acquiring continuous sensor data to determine ventilation quality, caregivers can devote their attention to actively managing the respiratory care of critically ill infants. Implementation of the disclosed subject matter can lead to enhanced respiratory outcomes in a substantial population of infants, with potential implications for improvement in long-term morbidity and mortality.

### Example Systems and Techniques for Predicting and Quantifying Delivered Amplitude, Frequency, and Mean Airway Pressure to Patients Receiving High Frequency Ventilation and Bubble CPAP

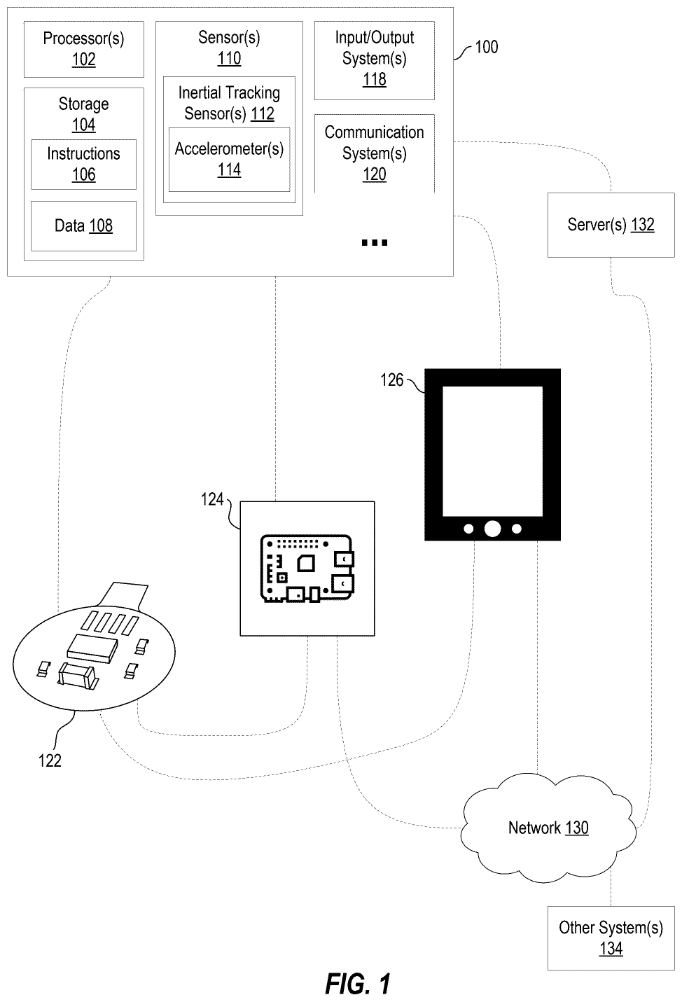

FIG. 1 illustrates example components 100 of example systems that may comprise or implement the disclosed embodiments. For example, the components illustrated in FIG. 1 include processor(s) 102, storage 104, sensor(s) 110, input/output system(s) 118 (I/O system(s) 118), and communication system(s) 120. Although FIG. 1 illustrates particular components, one will appreciate, in view of the present disclosure, that systems for implementing the disclosed embodiments may comprise any number of additional or alternative components (as indicated by the ellipsis).

The processor(s) 102 may comprise one or more sets of electronic circuitries that include any number of logic units, registers, and/or control units to facilitate the execution of computer-interpretable instructions (e.g., instructions that form a computer program). Such computer-interpretable instructions may be stored within storage 104. The storage 104 may comprise physical system memory (e.g., computer-readable recording media/memory) and may be volatile, non-volatile, or some combination thereof. Furthermore, storage 104 may comprise local storage, remote storage (e.g., accessible via communication system(s) 120 or otherwise), or some combination thereof. Additional details related to processors (e.g., processor(s) 102) and computer storage media (e.g., storage 104) will be provided hereinafter.

In some implementations, the processor(s) 102 may comprise or be configurable to execute any combination of software and/or hardware components that are operable to facilitate processing using machine learning models or other artificial intelligence-based structures/architectures. For example, processor(s) 102 may comprise and/or utilize hardware components or computer-executable instructions operable to carry out function blocks and/or processing layers configured in the form of, by way of non-limiting example, single-layer neural networks, feed forward neural networks, radial basis function networks, deep feed-forward networks, recurrent neural networks, long-short term memory (LSTM) networks, gated recurrent units, autoencoder neural networks, variational autoencoders, denoising autoencoders, sparse autoencoders, Markov chains, Hopfield neural networks, Boltzmann machine networks, restricted Boltzmann machine networks, deep belief networks, deep convolutional networks (or convolutional neural networks), deconvolutional neural networks, deep convolutional inverse graphics networks, generative adversarial networks, liquid state machines, extreme learning machines, echo state networks, deep residual networks, Kohonen networks, support vector machines, neural Turing machines, and/or others.

As will be described in more detail, the processor(s) 102 may be configured to execute instructions 106 stored within storage 104 to perform certain actions. The actions may rely at least in part on data 108 stored on storage 104 in a volatile and/or non-volatile manner.

In some instances, the actions may rely at least in part on communication system(s) 120 for receiving data from remote system(s), which may include, for example, separate systems or devices, sensors, servers, cloud resources/services, and/or others. The communications system(s) 120 may comprise any combination of software or hardware components that are operable to facilitate communication between on-system components/devices and/or with off-system components/devices. For example, the communications system(s) 120 may comprise ports, buses, or other physical connection apparatuses for communicating with other devices/components. Additionally, or alternatively, the communications system(s) 120 may comprise systems/components operable to communicate wirelessly with external systems and/or devices through any suitable communication channel(s), such as, by way of non-limiting example, Bluetooth, ultra-wideband, WLAN (e.g., Wi-Fi), infrared communication, and/or others.

FIG. 1 illustrates that a system for implementing the disclosed embodiments may comprise or be in communication with I/O system(s) 118. I/O system(s) 118 may include any type of input or output device such as, by way of non-limiting example, a touch screen, a mouse, a keyboard, a controller, a speaker, user interfaces, and/or others, without limitation. For example, the I/O system(s) 118 may include a display system that may comprise any number of display panels, optics, laser scanning display assemblies, and/or other components.

Furthermore, FIG. 1 illustrates that a system for implementing the disclosed embodiments may comprise or be in communication with sensor(s) 110. Sensor(s) 110 may comprise any device for capturing or measuring data representative of perceivable or detectable phenomena. By way of non-limiting example, the sensor(s) 110 may comprise one or more inertial tracking sensors (e.g., inertial tracking sensor(s) 112) such as one or more accelerometers (e.g., accelerometer(s) 114), thermometers, barometers, magnetometers, gyroscopes, microphones, image sensors, and/or others. Inertial tracking sensor(s) 112 may be configured to measure various kinematic properties of an object, such as acceleration, velocity, etc. Accelerometer(s) 114 can be configured to measure acceleration or the rate of change of velocity of an object along one or more movement axes (e.g., orthogonal axes, or X, Y, and Z axes). The accelerometer(s) 114 can be configured to output an electrical signal proportional to the acceleration they are measuring, and a separate signal can be output for each axes along which acceleration is measured. The accelerometer(s) 114 can utilize piezoelectric, capacitive, microelectromechanical systems (MEMS), or other sensing technology.

One or more of the components 100 shown in FIG. 1 and discussed hereinabove may be implemented in various types and/or combinations of systems/devices (as indicated by the dashed lines extending from the components 100 to various devices in FIG. 1). For example, any number of the components 100 discussed hereinabove with reference to FIG. 1 may be implemented in association with a ventilation quality sensor 122, which may comprise sensor(s) 110 (e.g., inertial tracking sensor(s) 112 such as accelerometer(s) 114) configured to capture acceleration data associated with a part of a subject (e.g., a chest of an infant) or a component of a ventilation device (e.g., a bubble chamber of a bCPAP).

In the example of FIG. 1, the ventilation quality sensor 122 comprises a flexible printed circuit board substrate, on which the accelerometer(s) 114 is/are affixed. The ventilation quality sensor 122 can comprise a small radius (e.g., within a range of 0.5-50 mm), which can improve adherence to the infant's chest while minimizing interference with clinical procedures. One will appreciate, in view of the present disclosure, that other forms/formats of a ventilation quality sensor 122 are within the scope of the disclosed subject matter. Inexpensive and readily available accelerometer(s) 114 can have a dynamic range high enough to accommodate measurement of chest wall vibrations and/or bCPAP bubbling, which can make the ventilation quality sensor 122 affordable and scalable in varied resourced settings and communities.

Although examples discussed herein focus, in at least some respects, on utilizing a ventilation quality sensor 122 with accelerometer(s) 114 to acquire and process acceleration data to determine chest wiggle, bCPAP bubbling quality (e.g., consistency of bubbles, frequency of bubbles, strength or intensity of pressure fluctuations created by the bubbles), and/or other outputs, a ventilation quality sensor 122 may comprise additional sensor(s) 110 to acquire additional sensor data that can supplement (or even replace) the acceleration data (e.g., to improve prediction/output robustness). For instance, a ventilation quality sensor 122 can comprise a microphone to obtain audio data, which can indicate bubbling quality of a bCPAP (e.g., in addition to acceleration data). In one example implementation, a ventilation quality sensor 122 that includes one or more microphones can be placed on one or more components of a respiratory support device, such as a bCPAP device. The microphone(s) can acquire audio data, which may capture sounds produced by the bubbling of the bCPAP device. The audio data capturing such sounds may be used as an additional (or alternative) input for generating ventilation quality output or predicted respiratory support device setting output. For instance, the audio data may be processed (potentially in conjunction with acceleration data acquired by the ventilation quality sensor) using one or more AI modules to generate the ventilation quality output or the predicted respiratory support device setting output. In some instances, the audio data is processed by one or more separate AI modules (e.g., different from the AI module(s) that process the acceleration data), and the output of the different AI modules may be aggregated, further processed, and/or used to modify one another to produce one or more output labels. As another example, a ventilation quality sensor system can include one or more image sensors (e.g., positioned on the ventilation quality sensor 122), which can capture image data that can provide augmentation of the acceleration data to determine chest wiggle (or can provide an alternative basis for determining chest wiggle) via one or more AI modules.

The ventilation quality sensor 122 may comprise processor(s) 102 and/or storage 104 associated with operation of the sensor(s) 110 for capturing the acceleration data (and/or other sensor data). In some instances, the ventilation quality sensor 122 is connected to one or more additional devices via communication system(s) 120 to facilitate control of or communication with the sensor(s) 110 of the ventilation quality sensor 122 (e.g., leveraging the processor(s) 102 and/or the storage 104 of the additional device(s)). For instance, the ventilation quality sensor 122 can be connected to a dedicated computing device 124, a tablet 126, or another type of computing device/system adapted for a patient care environment (as indicated in FIG. 1 by the dashed lines extending from the ventilation quality sensor 122 to the dedicated computing device 124 and the tablet 126). In some implementations, the dedicated computing device 124 comprises an inexpensive and readily available single-board computer that supports integration with accelerometers and/or other sensors, such as a Raspberry Pi. The dedicated computing device 124 can enable real-time data visualization and device control. The dedicated computing device 124 can be compact and attached to a convenient surface anywhere near the subject's incubator within a NICU (within range of a standard power outlet). Such a sensor system (e.g., a ventilation quality sensor 122 connected to a dedicated computing device 124) can be unobtrusive enough to blend in with a NICU environment and workflow.

In some implementations, the dedicated computing device 124 and/or any other device(s) associated with the ventilation quality sensor 122 can be configured to present an alarm and/or notification (e.g., an audible, visible, or even tactile alarm and/or notification) in response to certain conditions being satisfied, as will be described in more detail hereinafter.

Sensor data acquired via the ventilation quality sensor 122 can be received by the connected device (e.g., the dedicated computing device 124) for processing and/or transmission to other devices. For example, FIG. 1 shows that the dedicated computing device 124, and the tablet 126, and/or the other system(s) 134 may be in communication with a network 130. The network may comprise one or more links that enable the transport of information between and/or among systems, modules, and/or devices. FIG. 1 also illustrates server(s) 132 in communication with the network 130. The server(s) 132 may comprise any of the components 100 discussed hereinabove with reference to FIG. 1, such as processor(s) 102, storage 104, I/O system(s) 118, communication system(s) 120, etc. Any systems or devices shown or described with reference to FIG. 1 (e.g., the ventilation quality sensor 122, the dedicated computing device 124, the tablet 126, the other system(s) 134, the server(s) 132, or other systems) may be configured to receive, process, store, and/or display the sensor data acquired via the ventilation quality sensor 122 and/or information based thereon.

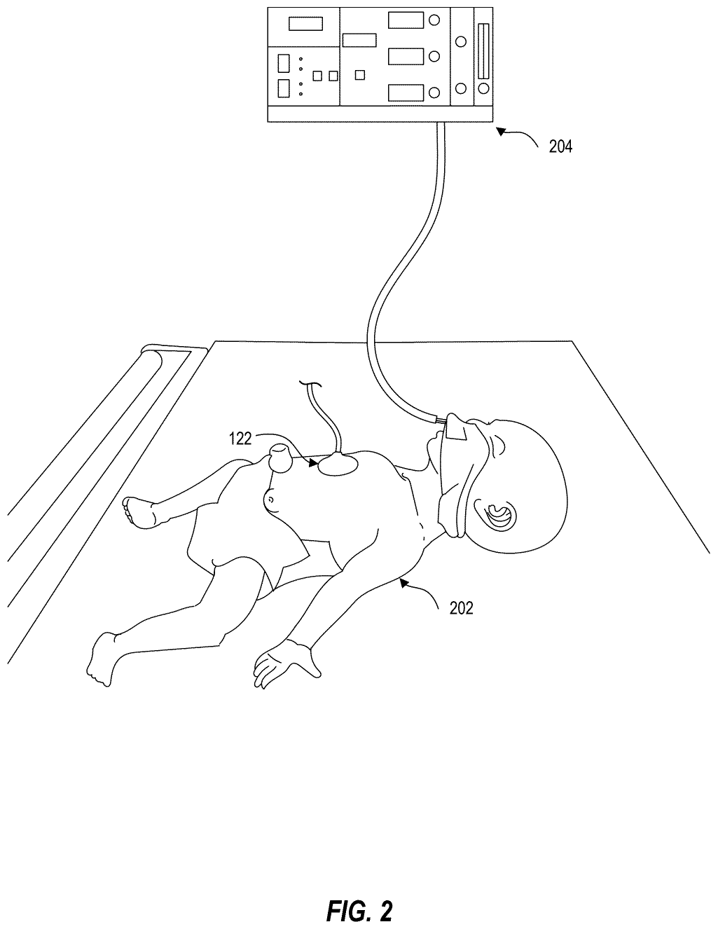

FIG. 2 illustrates a ventilation quality sensor 122 connected to a life-like micro-preemie model 202 for an average 25-week extremely low birth weight neonate, which includes molded-in lungs that produce realistic chest rise when ventilated by the mouth. FIG. 2 shows model 202 connected to a high frequency oscillatory ventilator 204 (HFOV). The model 202 can produce realistic chest wiggle under operation of the HFOV 204. In a clinical environment, with real infants, it can be difficult to visually observe chest wiggle (e.g., due to the size of the subject, room lighting artifacts, etc.). With the ventilation quality sensor 122 connected to the chest of an infant (as demonstrated in FIG. 2), the ventilation quality sensor 122 can be used to acquire acceleration data measuring the vibrations of the chest wall of the infant (e.g., in three orthogonal axes) to produce a real-time stream of data that can objectively measure chest wiggle of the infant. By quantifying chest wiggle through the use of a sensor, a ventilation quality sensor 122 can mitigate human errors inherent in subjective assessment of chest wiggle appearance.

In the example shown in FIG. 2, the ventilation quality sensor 122 is connected to the model 202 with an adhesive layer on the underside of the ventilation quality sensor 122. Other means for connecting a ventilation quality sensor 122 to a target object (e.g., a patient, a model of a patient, a component of a respiratory support device) may be utilized in accordance with the present disclosure (e.g., a transparent dressing such as Tegaderm, hydrocolloid strips, or other skin-friendly adhesives).

As noted above, in addition to objectively measuring chest wiggle in infants, a ventilation quality sensor 122 can be versatilely configured to additionally serve as a monitor for continual assessment of bubbling quality of bCPAPs. Silent loss of bCPAP functionality due to, for example, dislodgement of mask apparatus connected to the infant or a leak in the bCPAP system, can go unnoticed in a noisy NICU room or a room with multiple operational bCPAPs.

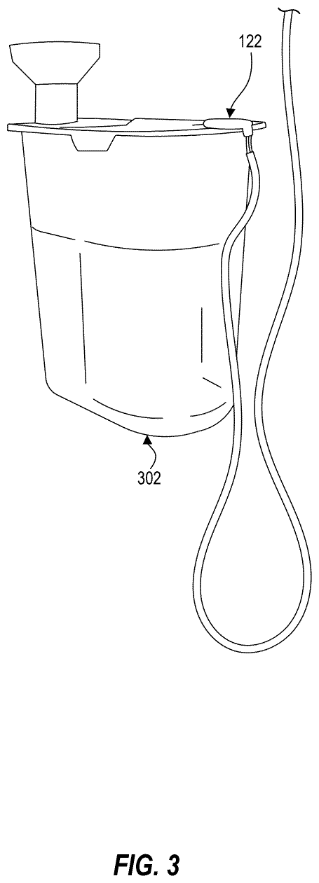

FIG. 3 illustrates a ventilation quality sensor 122 connected to a water/bubble chamber 302 of a bCPAP. The ventilation quality sensor 122 can be calibrated to sense loss or reduction of vibration of the water/bubble chamber 302. The ventilation quality sensor 122 can then acquire acceleration data to facilitate continual assessment of bubbling quality of bCPAPs. In some implementations, an alert can be triggered in response to detecting loss or reduction of vibration of the water/bubble chamber 302, which can enable the ventilation quality sensor 122 to detect silent loss of bCPAP functionality.

In some implementations, a ventilation quality sensor system (e.g., including a ventilation quality sensor 122 and a connected device, such as a dedicated computing device 124) utilizes an application of deep learning techniques to analyze timeseries data from the accelerometer(s) 114 of the ventilation quality sensor 122 to quantify or classify chest wiggle, bCPAP bubbling or ventilation quality, HFV quality, and/or other clinical events. For instance, acceleration data from the ventilation quality sensor 122 (or features extracted from the acceleration data) can be input to one or more AI modules that process the input data and output a wiggle classification or label (e.g., categorizing the wiggle quality as poor, moderate, or high). As another example, features extracted from acceleration data acquired by the ventilation quality sensor 122 can be provided as input to AI classifiers to categorize underlying clinical events (e.g., loss of bubbling, loss of wiggle, etc.).

Since it can be difficult for even experienced clinicians to describe the movement parameters that lead them to classify chest wiggle factor as ‘low quality’ or ‘high quality,’ disclosed embodiments can utilize automated techniques to extract features directly from the acceleration data. For example, a ventilation quality sensor system may utilize deep convolution networks to extract features from the acceleration data and may employ long short-term memory (LSTM) neural networks to classify the acceleration data into chest wiggle categories (or clinical event categories, such as loss or change in bubble characteristics).

In some implementations, movement features are derived from the raw sensor data (e.g., acceleration data) and those features are used to a) classify chest wiggle or bCPAP settings; and/or b) produce a quantified measure of chest wiggle using well established regression techniques. Features can be designed by experts or automatically derived from Deep learning neural networks when sufficient training data is available. Deep learning networks can vastly outperform expert-designed features whenever a) training data is available, and b) feature creation is not intuitive, and thus, complex. Traditional deep learning networks stack neural network layers (which extract progressively complex features from input signals) with a final set of fully connected layers that classify the output categorically or predict numerical values of a desired variable. LSTM networks can process sequential data with memory to handle temporal dependencies, enabling LSTM networks to extract patterns from an input signal that includes time-series data (e.g., the acceleration data acquired via a ventilation quality sensor 122). LSTM networks can thus be implemented in ventilation quality sensor systems to handle the 3-channel acceleration data (for 3 orthogonal axes) from ventilation quality sensors 122.

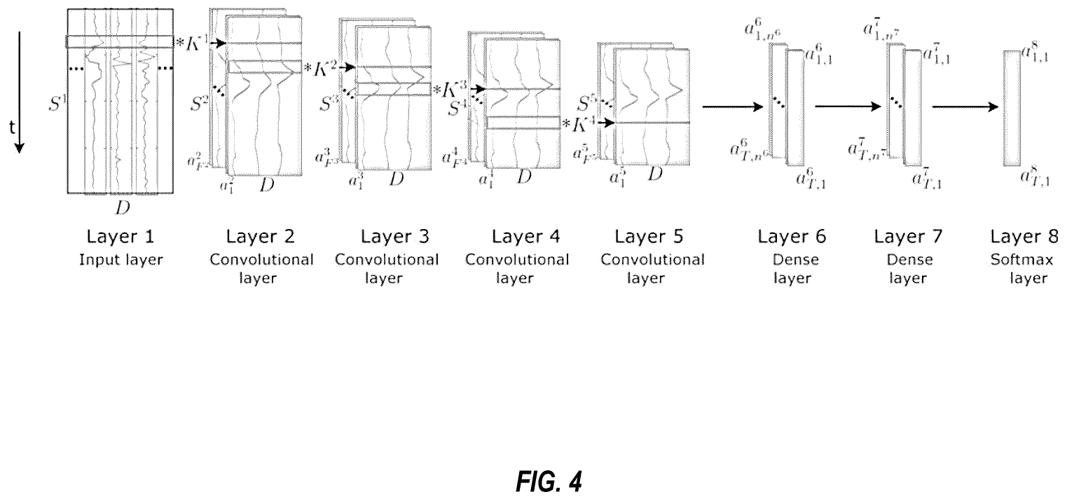

FIG. 4 provides a schematic of an AI module/model 400 (or simply “model 400”) that is configured to receive 3-channel acceleration data and output a category/classification label. The model can be implemented, by way of non-limiting example, using python scripts that use the Theano python library with an underlying open-source toolkit, Lasagne, which provides deep learning support.

As shown in FIG. 4, the input signal (comprising three channels) enters the model 400 at the left, and the final output layer (Layer 8) classifies the input signal as belonging to one of N categories. A temporal window of any length may be used to define the size of the input at a given instance. For example, where a temporal window length of 24 samples is used, the input may comprise 72 numbers (24 samples for each of the 3 sensor channels) comprising acceleration values that can be fed into the model 400. Continuing with the foregoing example, the output at Layer 5 may comprise 192 ‘features’ that can be conceptualized as patterns that have been derived from the input signals. The features (output at Layer 5) are then fed into two LSTM layers (Layers 6 & 7) that retain temporal memory of the input signals.

In some implementations, a model similar to model 400 may be trained to classify ventilator settings of a HFV device in response to acceleration data input (representing infant chest wiggle). The difference between the ventilator setting classifications output by the model and the dialed-in ventilator settings at an HFV device may be used as an objective basis for determining chest wiggle quality or ventilation quality delivered by the HFV device.

The following discussion refers to experimental results. It shall be noted that the experiments giving rise to the following results were performed under specific conditions using a specific embodiment or embodiments; accordingly, neither these experiments nor their results shall be used to limit the scope of the disclosed subject matter.

Experiments were conducted by acquiring vibration data from the infant mannequin (e.g., model 202) with the ventilation quality sensor 122 affixed to the chest (using Neobond) and the mannequin connected to a HFOV (e.g., HFOV 204). Five sets of data were acquired with various settings for three HFOV variables: frequency (F), amplitude (A), and mean airway pressure (MAP). For example, Set 1 represents the following ventilator values: F:15, A:30, MAP:15. The five sets represent common HFOV settings employed in the care of NICU infants. Each set represents about 15 minutes of data acquisition for a total of 4385 seconds (131, 550 samples, with 30 samples being collected per second) across all five settings of the ventilator. The 131,500 input samples, representing the five ventilator settings, were first converted into windowed samples by sliding a 24-sample window across the three channels of data, resulting in 10956 samples (where each sample contained 24×3=72 numbers). The samples were split into a training samples (80%) and test or validation samples (20%). The LSTM network (e.g., with a structure corresponding to model 400) was trained on the training samples (8764) and was tested on the remaining (2192) samples. After training, the LSTM network correctly classified the ventilator settings of the test samples with 98.76% accuracy, thus demonstrating the ability of a ventilation quality sensor system to infer ventilator settings from the observed vibrations at the chest.

In some implementations, chest wiggle quality or ventilation quality delivered by an HFV device is determined using inputs based on (i) the classified ventilator settings output by the LSTM network and (ii) the dialed-in ventilator settings of the HFV device. In some instances, large differences between the classified ventilator settings output by the LSTM network and the dialed-in ventilator settings of the HFV device may indicate low chest wiggle quality or ventilation quality delivered by the HFV device.

In some embodiments, a model similar to model 400 may be trained to classify column depth of a bCPAP device in response to acceleration data input (representing vibrations of the bubble chamber of the bCPAP device). The difference between the column depth bCPAP classifications output by the model and the actual column depth of a bCPAP device may be used as an objective basis for determining ventilation quality delivered by the bCPAP device.

The experimental process discussed above for training the LSTM model to infer ventilator settings from observed vibrations at the chest was repeated for bCPAP, with the ventilation quality sensor 122 placed on a bubble chamber (e.g., bubble chamber 302) and with sensor data being acquired for two different bCPAP column depth settings: 8 cm and 5 cm of pressure, obtaining 30 minutes of data for each setting. The trained LSTM network classified the bCPAP column depth settings with 100% accuracy. In some implementations, ventilation quality delivered by a bCPAP device is determined using inputs based on (i) the classified column depth setting output by the LSTM network and (ii) the actual column depth setting of the bCPAP device. In some instances, large differences between the classified column depth setting output by the LSTM network and the actual column depth settings of the bCPAP device may indicate low ventilation quality delivered by the bCPAP device.

A model similar to model 400 may be trained to output chest wiggle quality labels/categorizations in response to acceleration data input. For instance, training data comprising acceleration data (acquired via a ventilation quality sensor 122) and expert-labeled chest wiggle quality categorization data (e.g., labeling chest wiggle quality as poor, moderate, or high) may be used to train and validate the model 400 to enable the model 400, at inference, to receive acceleration data, derive features therefrom (e.g., at Layer 5), and process the features to classify/rank chest wiggle quality.

In some implementations, a feature extraction module (e.g., similar to Layers 1 through 5 of model 400) may be used in conjunction with regression techniques to predict or quantify ventilator settings of an HFV device in response to acceleration data input (representing infant chest wiggle). The difference between the ventilator setting predictions/quantifications output by the model and the dialed-in ventilator settings at an HFV device may be used as an objective basis for determining chest wiggle quality or ventilation quality delivered by the HFV device.

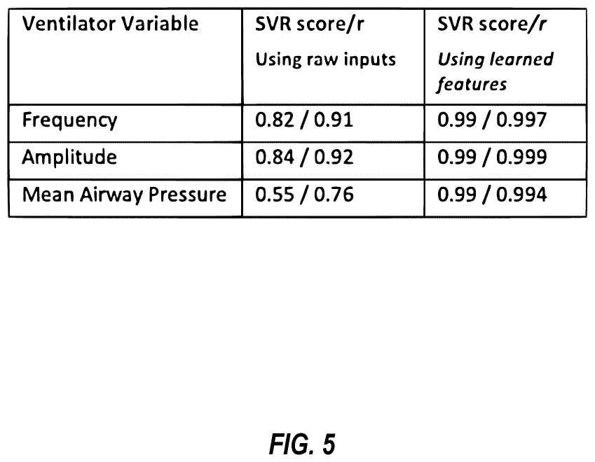

In one experiment, three different Support Vector Regressors (SVR) were trained to predict each of the three ventilator settings from the same ventilation quality sensor 122 input data stream (e.g., acceleration data). The SVRs can use, as input, the raw accelerometer values (at Layer 1 of the model 400) and, additionally or alternatively, features automatically derived from the raw accelerometer values (at Layer 5 of the model 400). The feature output comprised 192 features, which may be conceptualized as higher-level patterns that the network extracts from raw acceleration values. As shown in FIG. 5, the SVR predictions were evaluated using two metrics: SVR score and Pearson correlation coefficient (r). The SVR score indicates how well the regressor predicted the output, with values ranging from −1 to −1, with a value of 1 indicating perfect prediction. The correlation coefficient indicates how well the trend in the predicted output matches the trend in true values. As shown in FIG. 5, the regression metrics improve significantly when learned features are used instead of raw sensor data.

In some implementations, chest wiggle quality or ventilation quality delivered by an HFV device is determined using inputs based on (i) the predicted/quantified ventilator settings output by the SVR regressor(s) and (ii) the dialed-in ventilator settings of the HFV device.

In some embodiments, a feature extraction module (e.g., similar to Layers 1 through 5 of model 400) may be used in conjunction with regression techniques to predict or quantify column depth of a bCPAP device in response to acceleration data input (representing vibrations of the bubble chamber of the bCPAP device). For instance, the ventilation quality sensor 122 may be placed on the chamber of a bCPAP device (e.g., bubble chamber 302), and one or more SVR regressors may be trained to predict the bCPAP level (column depth in the water chamber, which corresponds to mean airway pressure in HFV). The difference between the column depth predictions/quantifications output by the model and the actual column depth of a bCPAP device may be used as an objective basis for determining ventilation quality delivered by the bCPAP device. In some implementations, ventilation quality delivered by a bCPAP device is determined using inputs based on (i) the predicted/quantified column depth setting output by the SVR regressor(s) and (ii) the actual column depth setting of the bCPAP device.

Infant chest wiggle quality, HFV quality, and/or bCPAP ventilation or bubble quality, as indicated by a ventilation quality sensor system, can be used by the clinician, in tandem with other non-real-time measures such as arterial blood gas (ABG) to proactively manage respiratory care of infants in a NICU.

The features extracted by the deep convolution networks (e.g., Layer 5 of model 400) can additionally or alternatively be used to predict changes in ventilation metrics such as SpO2 and PaCO2. Such predicted changes in ventilation metrics can provide a basis for alerting clinicians to deterioration of respiratory function prior to current data streams and lab tests indicating such a trend. For example, to predict SpO2 or PaCO2 values for infants on HFV, training data including acceleration data (e.g., from ventilation quality sensors 122 placed on infant chests), FiO2 measurements, SpO2 measurements, tcPCO2 measurements, HFV amplitude, HFV frequency, HFV MAP, and/or chest wiggle factor categorization (expert-labeled poor, moderate, or high) may be used to train LSTM models to perform classification of chest wiggle values and to produce features that can drive the SVRs to predict quantitative values for SpO2 and/or PaCO2 based on the current and most recent chest wiggle values. To predict SpO2 or PaCO2 values for infants on bCPAP, the training data may include acceleration data (e.g., from ventilation quality sensors 122 placed on infant chests and/or on bCPAP bubble chambers), FiO2 measurements, SpO2 measurements, tcPCO2 measurements, bCPAP level, and/or bCPAP flow. The SVR regressors may be trained to predict quantitative values for SpO2 and/or PaCO2 based on features extracted from the acceleration data and/or bCPAP level and/or bCPAP flow values.

In some implementations, the LSTM and SVR model building processes discussed hereinabove may employ the leave one subject out technique to train the models on all but one subject, holding out the remaining subject data for validation, and repeating the process across all subjects. Model performance measures, derived using the leave one subject out technique, can produce realistic estimates of performance in a clinical setting.

Although examples discussed herein focus, in at least some respects, on utilizing LSTM networks/layers to facilitate determination of HFV ventilation quality, chest wiggle quality, or bCPAP ventilation quality, other types of networks/layers may be used in accordance with the present disclosure (e.g., gated recurrent units (GRUs), echo state networks (ESNs), and/or others). Furthermore, although examples discussed herein focus, in at least some respects, on utilizing SVR techniques to facilitate determination of HFV ventilation quality, bCPAP ventilation quality, SpO2 values, or PaCO2 values, other regression techniques may be used in accordance with the present disclosure (e.g., decision trees, gradient boosting regressors, Huber regression, and/or others).

To achieve the AI-based functionality of a ventilation quality sensor system, as described above, the AI models (e.g., machine learning models) may be trained and configured as executable packages (e.g., Docker images), which may be deployed in on-premises computing systems (e.g., server(s) 132). The deployed executable packages may be accessible via an API call to facilitate processing of sensor data for requesting devices (e.g., tablet 126, other system(s) 134) to guide NICU care decisions. Additionally, or alternatively, the trained AI models may be ported to one or more dedicated computing devices 124 in one or more clinical environments (e.g., NICUs). In some instances, the dedicated computing device(s) 124 may be implemented as touch-operated user interfaces in the clinical environment(s). The dedicated computing device(s) 124 in the clinical environment(s) may be connected to one or more ventilation quality sensors 122 to enable onboard inferencing that can reduce latency and avoid reliance on persistent network/internet connections.

As noted above, various systems or devices (e.g., the ventilation quality sensor 122, the dedicated computing device 124, the tablet 126, the other system(s) 134, the server(s) 132, or other systems) may be configured to receive, process, store, and/or display the sensor data acquired via the ventilation quality sensor 122 and/or information based thereon. A ventilation quality sensor system can be constructed as a highly scalable cloud platform augmented with a local desktop client application running on the dedicated computing device 124 for real-time visualization and device control. A web frontend, accessible from any modern browser (e.g., on the tablet 126, the other system(s) 134, etc.) can provide an administration dashboard from which the clinician can manage multiple ventilation quality sensors 122 or associated devices and visualize the metrics.

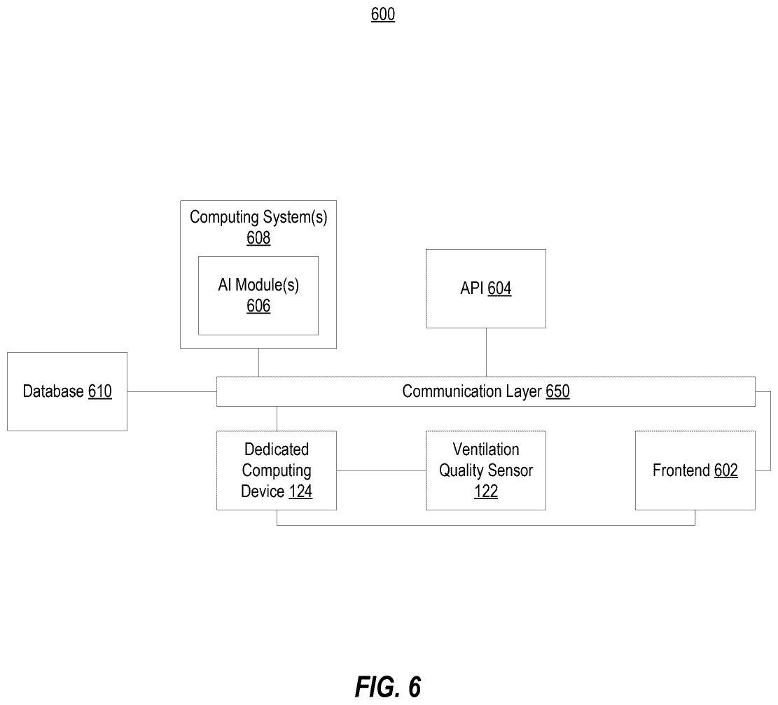

Advantageously, a ventilation quality sensor system can be built on commonly available open-source toolkits and commercially available hardware. FIG. 6 provides a conceptual representation of an example architecture 600 for example components of a ventilation quality sensor system, including a communication layer 650 for facilitating communication among various components. A web or application based frontend 602 can provide administrative and human subject study functions and overview of device status, and can be built with React (an open-source JavaScript library from Meta) or another library. The API 604 for accessing executable package(s) (e.g., the AI module(s) 606 in FIG. 6) deployed on the computing system(s) 608 (on-premises or off-premises) can, in some implementations, be built on NodeJS technology and hosted as a cloud service (e.g., Heroku.com). The platform can adopt a distributed architecture with loose coupling between the modules, linked through sockets (e.g., socket.io) and queues (e.g., Amazon Simple Queue Service). Timeseries data, acquired by the ventilation quality sensor 122 can be processed onboard the dedicated computing device 124 and can be stored in a database 610 (e.g., a cloud-hosted NoSQL database such as MongoDB), which can be optimized for storage and rapid retrieval of time-series data. The dedicated computing device 124 hardware can run a set of python scripts to gather data (e.g., at 30 samples/sec) from the accelerometer(s) 114 of the ventilation quality sensor 122 along three axes and persist them via the API 604 to a backend storage. This data can then be downloaded by the computing system(s) 608 to train the AI module(s) 606 that quantify chest wiggle and/or quantify bubbling quality of a bCPAP device (and/or other ventilation quality outputs or predicted respiratory support device setting outputs described herein). Model training can be performed using on-premises machines (e.g., of the computing system(s) 608) to create the AI module(s) 606. At the bedside, the dedicated computing device 124 can be configured with a touchscreen display that has a single-board (or other type of) computer attached to the back, obviating the need for external keyboard and monitor to access the dedicated computing device 124 device in the NICU. Using socket technology, the admin can be allowed to remotely operate the dedicated computing device 124 device at the bedside. However, to reduce latencies and to visualize data at higher rates (e.g., 30 Hz), the dedicated computing device 124 can provide a simple user interface to operate the device and visualize the accelerometer outputs.

As described herein, a ventilation quality sensor system can be implemented in conjunction with bCPAP devices and/or HFV devices to facilitate monitoring of ventilation quality delivered to a patient. In some implementations, multiple AI modules are trained to infer ventilation quality outputs for different operational contexts. For instance, a ventilation quality sensor system can include a first set of one or more AI modules trained to infer chest wiggle labels (e.g., classification or quantification), oxygenation levels, CO2 levels, HFV device settings, and/or clinical events (e.g., loss of chest wiggle) when the acceleration data is obtained with the ventilation quality sensor 122 connected to the chest of a patient (or model of a patient). The same ventilation quality sensor system can additionally include a second set of one or more AI modules trained to infer bCPAP bubbling quality labels (e.g., classification or quantification), oxygenation levels, CO2 levels, bCPAP device settings (e.g., column depth), and/or clinical events (e.g., loss of bubbling) when the acceleration data is obtained with the ventilation quality sensor connected to the chamber of a bCPAP device.

Where multiple AI modules included in a ventilation quality sensor system, the ventilation quality sensor system can be configured to select which AI module(s) to use to infer ventilation quality output and/or predicted respiratory support device setting output. For example, a user interface fronted associated with the ventilation quality sensor system (e.g., on a dedicated computing device 124 or other device) can be configured to receive user input for indicating which AI module(s) to use for inference. For instance, the user input can indicate whether the ventilation quality sensor 122 is connected to a patient (or model of a patient) or a component of a respiratory support device (e.g., bCPAP chamber), which can signal to the ventilation quality sensor system which of the AI module(s) to use for inference. In some implementations, the ventilation quality sensor system is configured to pre-process acceleration data obtained via the ventilation quality sensor 122 to determine which AI module(s) to use for inference. For instance, the ventilation quality sensor system can be configured to perform feature extraction, signal processing (e.g., filtering, wavelet transform, cross-correlation), pattern recognition, profiling/template matching, machine learning techniques, and/or other techniques on the acceleration data to determine how the ventilation quality sensor 122 is connected and/or which AI module(s) to utilize for inference.

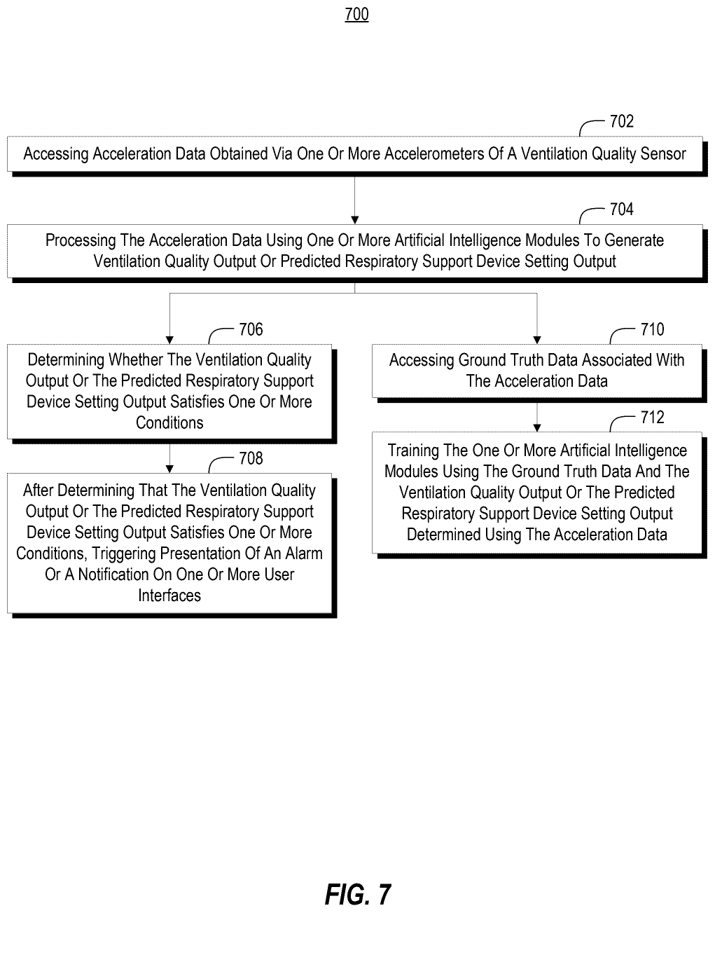

FIGS. 7 through 9 illustrate example flow diagrams depicting acts associated with the disclosed subject matter. Although the flow diagrams illustrate acts in a particular order, no specific ordering is required unless otherwise stated or unless performance of one act relies on completion of another. One or more of the acts shown and/or described in the flow diagrams may be omitted in some embodiments.

The acts described in association with the flow diagrams may be performed using one or more components of a ventilation quality sensor system, as described herein. For instance, various acts may be performed using processor(s) 102, storage 104, communication system(s) 120, and/or other components, which may be located on a dedicated computing device 124, server(s) 132, and/or other computing systems.

Act 702 of flow diagram 700 of FIG. 7 includes accessing acceleration data obtained via one or more accelerometers of a ventilation quality sensor. In some instances, the ventilation quality sensor comprises a flexible printed circuit board substrate, and the one or more accelerometers can be affixed to the flexible printed circuit board substrate. In some implementations, the ventilation quality sensor comprises a radius within a range of about 7 mm to about 30 mm. In some examples, the ventilation quality sensor comprises an adhesive for connecting the ventilation quality sensor to a human patient, a model of a human patient, or one or more components of a respiratory support device (e.g., a bCPAP device or an HFV device). In some embodiments, the acceleration data comprises 3-channel acceleration data, where each channel of the 3-channel acceleration data is associated with a respective movement axis (e.g., x, y, and z axes). In some instances, the acceleration data is obtained via the one or more accelerometers when the ventilation quality sensor is connected to a human patient or a model of a human patient (e.g., when operating in conjunction with an HFV device). In some implementations, the acceleration data is obtained via the one or more accelerometers when the ventilation quality sensor is connected to one or more components of a respiratory support device (e.g., a chamber of a bCPAP device).

Act 704 of flow diagram 700 includes processing the acceleration data using one or more artificial intelligence modules to generate ventilation quality output or predicted respiratory support device setting output. In some examples, the one or more artificial intelligence modules are configured to: (i) extract one or more features from the acceleration data; and (ii) use the one or more features to determine the ventilation quality output or the predicted respiratory support device setting output. In some embodiments, the ventilation quality output comprises one or more of: a chest wiggle classification (e.g., poor, moderate, or high), a chest wiggle quantification (e.g., a numerical chest wiggle score indicating chest wiggle quality), a bCPAP bubbling quality classification (e.g., a quality label for bubble consistency, bubble frequency, or pressure fluctuations caused by bubbles), a bCPAP bubbling quality quantification (e.g., a numerical bubbling score indicating quality of bubbling consistency, bubbling frequency, or pressure fluctuations caused by bubbles), an oxygenation level (e.g., a predicted oxygenation saturation measurement such as a predicted SpO2 measurement, or a predicted inspired oxygen measurement such as a predicted FiO2 measurement), a CO2 level (e.g., a predicted transcutaneous carbon dioxide pressure measurement, such as a predicted tcPCO2 measurement), or an indication of occurrence of one or more clinical events (e.g., loss of bCPAP bubbling, loss of chess wiggle).

In some instances, the predicted respiratory support device setting output comprises one or more of: one or more predicted operational settings for an HFV device (e.g., frequency, amplitude, MAP) or one or more predicted operational settings for a bCPAP device (e.g., column depth).

In some implementations, after performance of act 704, one or more components of a ventilation quality sensor system perform acts 706 and 708. Act 706 of flow diagram 700 includes determining whether the ventilation quality output or the predicted respiratory support device setting output satisfies one or more conditions. Act 708 of flow diagram 700 includes, after determining that the ventilation quality output or the predicted respiratory support device setting output satisfies one or more conditions, triggering presentation of an alarm or a notification on one or more user interfaces.

In some examples, the one or more conditions comprise one or more of: the ventilation quality output comprising a chest wiggle classification that corresponds to a predefined chest wiggle classification (e.g., poor), the ventilation quality output indicating a change in chest wiggle classification (e.g., changing from high to moderate or poor, or changing from moderate to poor), the ventilation quality output comprising a chest wiggle score that satisfies a predefined chest wiggle score threshold (e.g., a chest wiggle score being below a certain level), the ventilation quality output indicating a change in chest wiggle score that satisfies a predefined chest wiggle score change threshold (e.g., chest wiggle scores falling over time at a certain rate), the ventilation quality output comprising a bCPAP bubbling quality classification that corresponds to a predefined bCPAP bubbling quality classification (e.g., poor bubble consistency, bubble frequency, or pressure fluctuations caused by bubbles), the ventilation quality output indicating a change in bCPAP bubbling quality classification, the ventilation quality output comprising a bCPAP bubbling quality score that satisfies a predefined bCPAP bubbling quality score threshold (e.g., a bubbling quality score that indicates bubble consistency, bubble frequency, or pressure fluctuations caused by bubbles being below a certain level), the ventilation quality output indicating a change in bCPAP bubbling quality score that satisfies a predefined bCPAP bubbling quality score change threshold (e.g., bubbling quality scores falling over time at a certain rate), the ventilation quality output comprising an oxygenation level that satisfies a threshold oxygenation level, the ventilation quality output indicating a change in oxygenation level that satisfies a predefined oxygenation level change threshold, the ventilation quality output comprising a CO2 level that satisfies a threshold CO2 level, the ventilation quality output indicating a change in CO2 level that satisfies a predefined CO2 level change threshold, or the ventilation quality output indicating an occurrence of one or more clinical events (e.g., loss of bCPAP bubbling, loss of chest wiggle).

In some implementations, one or more components of a ventilation quality sensor system can be configured to (i) obtain operational settings of a respiratory support device (e.g., column depth for a bCPAP device, or frequency, amplitude, and/or MAP for an HFV device) and (ii) determine a difference (using regression techniques) between the obtained operational settings and the predicted respiratory support device setting output (e.g., predicted column depth for a bCPAP device, or predicted frequency, predicted amplitude, and/or predicted MAP for an HFV device). This difference satisfying a difference threshold can comprise a condition for triggering presentation of an alarm or a notification in accordance with act 708.

In some implementations, after performance of act 704, one or more components of a ventilation quality sensor system perform acts 710 and 712. Act 710 of flow diagram 700 includes accessing ground truth data associated with the acceleration data. In some instances, the ground truth data comprises one or more of: one or more chest wiggle ground truth labels, one or more bCPAP bubbling quality ground truth labels, one or more oxygenation level ground truth labels, one or more CO2 level ground truth labels, one or more clinical even occurrence ground truth labels, or one or more operational setting ground truth labels for a respirator support device (e.g., ground truth column depth for a bCPAP device, or ground truth frequency, amplitude, and/or MAP for an HFV device).

Act 712 of flow diagram 700 includes training the one or more artificial intelligence modules using the ground truth data and the ventilation quality output or the predicted respiratory support device setting output determined using the acceleration data. Any suitable supervised learning or other training techniques may be used to train the one or more artificial intelligence modules.

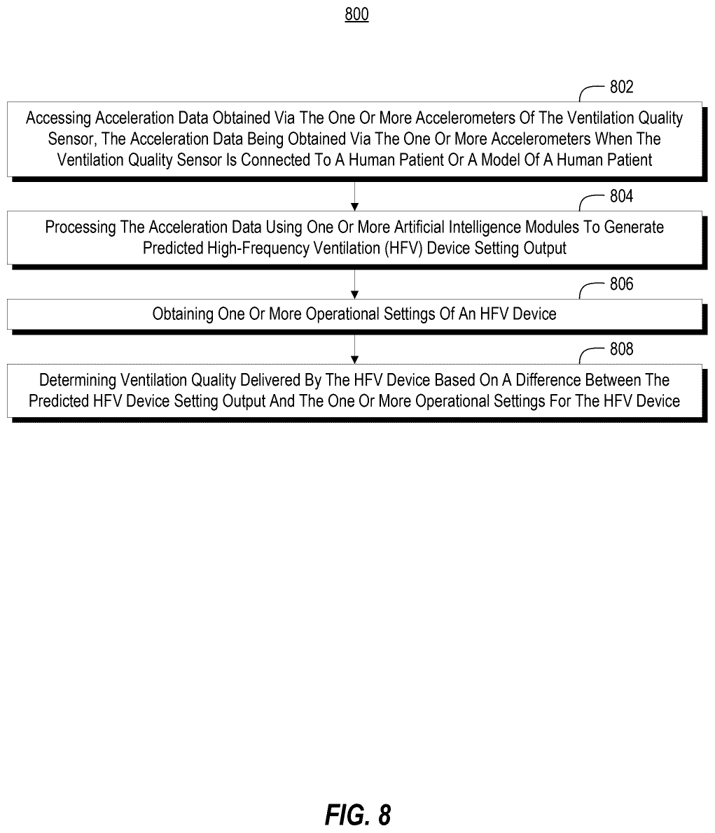

Act 802 of flow diagram 800 of FIG. 8 includes accessing acceleration data obtained via the one or more accelerometers of the ventilation quality sensor, the acceleration data being obtained via the one or more accelerometers when the ventilation quality sensor is connected to a human patient or a model of a human patient.

Act 804 of flow diagram 800 includes processing the acceleration data using one or more artificial intelligence modules to generate predicted high-frequency ventilation (HFV) device setting output. In some implementations, the one or more artificial intelligence modules are configured to: (i) extract one or more features from the acceleration data; and (ii) use the one or more features to determine the predicted HFV device setting output. In some examples, the one or more predicted operational settings for the HFV device comprise frequency, amplitude, or MAP.

Act 806 of flow diagram 800 includes obtaining one or more operational settings of an HFV device.

Act 808 of flow diagram 800 includes determining ventilation quality delivered by the HFV device based on a difference between the predicted HFV device setting output and the one or more operational settings for the HFV device.

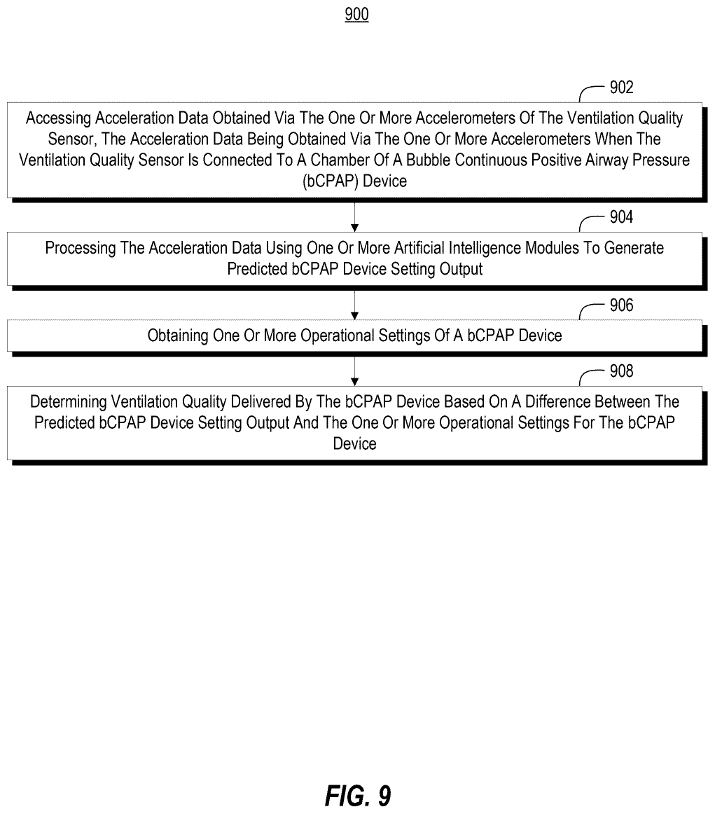

Act 902 of flow diagram 900 of FIG. 9 includes accessing acceleration data obtained via the one or more accelerometers of the ventilation quality sensor, the acceleration data being obtained via the one or more accelerometers when the ventilation quality sensor is connected to a chamber of a bubble continuous positive airway pressure (bCPAP) device.

Act 904 of flow diagram 900 includes processing the acceleration data using one or more artificial intelligence modules to generate predicted bCPAP device setting output. In some embodiments, the one or more artificial intelligence modules are configured to: (i) extract one or more features from the acceleration data; and (ii) use the one or more features to determine the predicted bCPAP device setting output. In some instances, the one or more predicted operational settings for the bCPAP device comprise column depth.

Act 906 of flow diagram 900 includes obtaining one or more operational settings of a bCPAP device.

Act 908 of flow diagram 900 includes determining ventilation quality delivered by the bCPAP device based on a difference between the predicted bCPAP device setting output and the one or more operational settings for the bCPAP device.

### Additional Details Related to Implementing the Disclosed Embodiments

Disclosed embodiments may comprise or utilize a special purpose or general-purpose computer including computer hardware, as discussed in greater detail below. Disclosed embodiments also include physical and other computer-readable media for carrying or storing computer-executable instructions and/or data structures. Such computer-readable media can be any available media that can be accessed by a general-purpose or special-purpose computer system. Computer-readable media that store computer-executable instructions in the form of data are one or more “physical computer storage media” or “hardware storage device(s)” or “computer-readable recording media.” Computer-readable media that merely carry computer-executable instructions without storing the computer-executable instructions are “transmission media.” Thus, by way of example and not limitation, the current embodiments can comprise at least two distinctly different kinds of computer-readable media: computer storage media and transmission media.

Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in hardware in the form of computer-executable instructions, data, or data structures and that can be accessed by a general-purpose or special-purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmission media can include a network and/or data links which can be used to carry program code in the form of computer-executable instructions or data structures and which can be accessed by a general purpose or special purpose computer. Combinations of the above are also included within the scope of computer-readable media.

Further, upon reaching various computer system components, program code in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Disclosed embodiments may comprise or utilize cloud computing. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, wearable devices, and the like. The invention may also be practiced in distributed system environments where multiple computer systems (e.g., local and remote systems), which are linked through a network (either by hardwired data links, wireless data links, or by a combination of hardwired and wireless data links), perform tasks. In a distributed system environment, program modules may be located in local and/or remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Application-specific Standard Products (ASSPs), System-on-a-chip systems (SOCs), Complex Programmable Logic Devices (CPLDs), central processing units (CPUs), graphics processing units (GPUs), and/or others.

As used herein, the terms “executable module,” “executable component,” “component,” “module,” or “engine” can refer to hardware processing units or to software objects, routines, or methods that may be executed on one or more computer systems. The different components, modules, engines, and services described herein may be implemented as objects or processors that execute on one or more computer systems (e.g., as separate threads).

One will also appreciate how any feature or operation disclosed herein may be combined with any one or combination of the other features and operations disclosed herein. Additionally, the content or feature in any one of the Figures may be combined or used in connection with any content or feature used in any of the other Figures. In this regard, the content disclosed in any one figure is not mutually exclusive and instead may be combinable with the content from any of the other Figures.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatuses disclosed herein may be made without departing from the scope of the disclosure or of the invention. Thus, while various aspects and embodiments have been disclosed herein, other aspects and embodiments are contemplated. All changes that come within the meaning and range of equivalency of the claims are to be embraced within their scope.

What is claimed is:

1. A ventilation quality sensor system, comprising:
a ventilation quality sensor comprising one or more accelerometers;
one or more processors; and
one or more computer-readable recording media that store instructions that are executable by the one or more processors to configure the ventilation quality sensor system to:
access acceleration data obtained via the one or more accelerometers of the ventilation quality sensor; and
process the acceleration data using one or more artificial intelligence modules to generate ventilation quality output or predicted respiratory support device setting output.

2. The ventilation quality sensor system of claim 1, wherein the ventilation quality sensor comprises a flexible printed circuit board substrate, and wherein the one or more accelerometers are affixed to the flexible printed circuit board substrate.

3. The ventilation quality sensor system of claim 1, wherein the ventilation quality sensor further comprises one or more microphones, and wherein audio data obtained via the one or more microphones is used as an input to generate or modify the ventilation quality output or the predicted respiratory support device setting output.

4. The ventilation quality sensor system of claim 2, wherein the ventilation quality sensor comprises an adhesive for connecting the ventilation quality sensor to a human patient, a model of a human patient, or one or more components of a respiratory support device.

5. The ventilation quality sensor system of claim 1, wherein the acceleration data comprises 3-channel acceleration data, wherein each channel of the 3-channel acceleration data is associated with a respective movement axis.

6. The ventilation quality sensor system of claim 1, wherein the acceleration data is obtained via the one or more accelerometers when the ventilation quality sensor is connected to a human patient or a model of a human patient.

7. The ventilation quality sensor system of claim 1, wherein the acceleration data is obtained via the one or more accelerometers when the ventilation quality sensor is connected to one or more components of a respiratory support device.

8. The ventilation quality sensor system of claim 7, wherein the one or more components of the respiratory support device comprise a chamber of a bubble continuous positive airway pressure (bCPAP) device.

9. The ventilation quality sensor system of claim 1, wherein the one or more artificial intelligence modules are configured to:
extract one or more features from the acceleration data; and
use the one or more features to determine the ventilation quality output or the predicted respiratory support device setting output.

10. The ventilation quality sensor system of claim 1, wherein the ventilation quality output comprises one or more of: a chest wiggle classification, a chest wiggle quantification, a bCPAP bubbling quality classification, a bCPAP bubbling quality quantification, an oxygenation level, a CO2 level, or an indication of occurrence of one or more clinical events.

11. The ventilation quality sensor system of claim 10, wherein the one or more clinical events comprise one or more of: loss of bCPAP bubbling or loss of chest wiggle.

12. The ventilation quality sensor system of claim 1, wherein the predicted respiratory support device setting output comprises one or more of: one or more predicted operational settings for a high-frequency ventilation (HFV) device or one or more predicted operational settings for a bCPAP device.

13. The ventilation quality sensor system of claim 12, wherein the one or more predicted operational settings for the HFV device comprise frequency, amplitude, or mean airway pressure (MAP), and wherein the one or more predicted operational settings for the bCPAP device comprise column depth.

14. The ventilation quality sensor system of claim 1, wherein the instructions are executable by the one or more processors to configure the ventilation quality sensor system to:
obtain one or more operational settings of a respiratory support device; and
determine a difference between the one or more operational settings of the respiratory support device and the predicted respiratory support device setting output, wherein the difference indicates ventilation quality delivered by the respiratory support device.

15. The ventilation quality sensor system of claim 1, wherein the instructions are executable by the one or more processors to configure the ventilation quality sensor system to:
determine whether the ventilation quality output or the predicted respiratory support device setting output satisfies one or more conditions; and
after determining that the ventilation quality output or the predicted respiratory support device setting output satisfies one or more conditions, trigger presentation of an alarm or a notification on one or more user interfaces.

16. The ventilation quality sensor system of claim 15, wherein the one or more conditions comprise one or more of:
the ventilation quality output comprising a chest wiggle classification that corresponds to a predefined chest wiggle classification;
the ventilation quality output indicating a change in chest wiggle classification;
the ventilation quality output comprising a chest wiggle score that satisfies a predefined chest wiggle score threshold;
the ventilation quality output indicating a change in chest wiggle score that satisfies a predefined chest wiggle score change threshold;
the ventilation quality output comprising a bCPAP bubbling quality classification that corresponds to a predefined bCPAP bubbling quality classification;
the ventilation quality output indicating a change in bCPAP bubbling quality classification;
the ventilation quality output comprising a bCPAP bubbling quality score that satisfies a predefined bCPAP bubbling quality score threshold;
the ventilation quality output indicating a change in bCPAP bubbling quality score that satisfies a predefined bCPAP bubbling quality score change threshold;
the ventilation quality output comprising an oxygenation level that satisfies a threshold oxygenation level;
the ventilation quality output indicating a change in oxygenation level that satisfies a predefined oxygenation level change threshold;
the ventilation quality output comprising a CO2 level that satisfies a threshold CO2 level;
the ventilation quality output indicating a change in CO2 level that satisfies a predefined CO2 level change threshold;
the ventilation quality output indicating an occurrence of one or more clinical events; or
a difference between (i) one or more operational settings of a respiratory support device and (ii) the predicted respiratory support device setting output satisfying one or more difference thresholds.

17. The ventilation quality sensor system of claim 1, wherein the instructions are executable by the one or more processors to configure the ventilation quality sensor system to:
access ground truth data associated with the acceleration data; and
train the one or more artificial intelligence modules using the ground truth data and the ventilation quality output or the predicted respiratory support device setting output determined using the acceleration data.

18. The ventilation quality sensor system of claim 17, wherein the ground truth data comprises one or more of: one or more chest wiggle ground truth labels, one or more bCPAP bubbling quality ground truth labels, one or more oxygenation level ground truth labels, one or more CO2 level ground truth labels, one or more clinical even occurrence ground truth labels, or one or more operational setting ground truth labels for a respirator support device.

19. A ventilation quality sensor system, comprising:
a ventilation quality sensor comprising one or more accelerometers;
one or more processors; and
one or more computer-readable recording media that store instructions that are executable by the one or more processors to configure the ventilation quality sensor system to:
access acceleration data obtained via the one or more accelerometers of the ventilation quality sensor, the acceleration data being obtained via the one or more accelerometers when the ventilation quality sensor is connected to a human patient or a model of a human patient;
process the acceleration data using one or more artificial intelligence modules to generate predicted high-frequency ventilation (HFV) device setting output;
obtain one or more operational settings of an HFV device; and
determine ventilation quality delivered by the HFV device based on a difference between the predicted HFV device setting output and the one or more operational settings for the HFV device.

20. A ventilation quality sensor system, comprising:
a ventilation quality sensor comprising one or more accelerometers;
one or more processors; and
one or more computer-readable recording media that store instructions that are executable by the one or more processors to configure the ventilation quality sensor system to:
access acceleration data obtained via the one or more accelerometers of the ventilation quality sensor, the acceleration data being obtained via the one or more accelerometers when the ventilation quality sensor is connected to a chamber of a bubble continuous positive airway pressure (bCPAP) device;
process the acceleration data using one or more artificial intelligence modules to generate predicted bCPAP device setting output;
obtain one or more operational settings of a bCPAP device; and
determine ventilation quality delivered by the bCPAP device based on a difference between the predicted bCPAP device setting output and the one or more operational settings for the bCPAP device.
